(19) United States
(12) Patent Application Publication
(10) Pub. No.: US 2010/0240028 A1
(43) Pub. Date: Sep. 23, 2010

(54) METHOD TO IMPROVE QUALITY OF MICROARRAYS BY CONTROLLING EVAPORATION

(75) Inventors: Alexander N. Perov, Woodridge, IL (US); Darrell P. Chandler, Yorkville, IL (US)

Correspondence Address: BARNES & THORNBURG LLP, P.O. BOX 2786, CHICAGO, IL, 60690-2786, US

(73) Assignee: THE UNIVERSITY OF CHICAGO, Chicago, IL (US)

(21) Appl. No.: 11/466,717

(22) Filed: Aug. 23, 2006

Related U.S. Application Data
(60) Provisional application No. 60/710,416, filed on Aug. 23, 2005.

(51) Int. Cl. C12Q 1/68

(52) U.S. Cl. 435/6; 438/22; 438/48; 435/287.2; 422/130

(57) ABSTRACT

Methods and compositions described here minimize loss due to evaporation during the process of preparing gel-element microarrays; maintain uniform gel density; minimize irregular spontaneous polymerization; and provide conditions to obtain reproducible and consistent gel-array elements. The materials and methods described herein present techniques for restoring the original composition of the droplets dispensed on the microarray substrates prior to and during the step of ultraviolet light polymerization.

### BACKGROUND

This application claims priority from copending U.S. Ser. No. 60/710,416 filed Aug. 23, 2005. This invention was partially conceived under Contract No. W-31-109-ENG-38 between the United States Department of Energy and the University of Chicago representing Argonne National Laboratory.

A method to improve quality of microarrays is to maintain atmospheric equilibrium of the environment during microarray preparation, which means there is no net evaporation.

To form microarrays, which on substrates are referred to as microchips, biochips and the like, compositions of molecules such as nucleic acids and proteins are immobilized in carriers, for example, in polymers such as gels. Various methods are available to transfer or affix the compositions of molecules and carriers to substrates. Suitable substrates include glass slides, plastic slides or films, and beads.

In gel-based microarray fabrication (microchips, biochips), gel is synthesized in various ways, including by a spatially selective (mask-guided) ultraviolet (UV) polymerization in a thin continuous layer (Mirzabekov et al., U.S. Pat. No. 6,465,174) and by contact printing of a liquid polymerization composition with a robotic arrayer and subsequent UV polymerization.

U.S. Pat. Pub. No. US20040053298 (Mirzabekov et al.) describes methods and compositions to manufacture gel-based arrays by immobilization of molecules in polymer carriers including oligonucleotides, proteins, nucleic acids, and any other molecules whose structure includes active groups such as amino and sulfhydryl groups. The arrays may be produced by contact printing with a robotic arrayer which has flexibility, but has problems. For example, maintaining the correct composition of the polymerization mixture, which may change with time due to evaporation of water and other relatively volatile components, is an issue. Before polymerization, droplets of a suitable composition of molecules are held in a container, e.g. a source plate. Contact printing of gel-based microarrays involves droplets with volumes of, for example, 1 nl, formed on microarray substrates. Because of the small volume, the droplets are generally depleted of acrylamide (monomer) due to evaporation, in less than one hour. Such changes may lead to irreproducibility of the gel compositions. There are effects of evaporation both on samples in the wells of a source microplate (possible spontaneous polymerization) and in the droplets (loss of acrylamide) obtained from the source. When acrylamide is lost, the droplets lose their ability to polymerize. On the other hand, the concentration of acrylamide in the wells of the source plate may substantially increase over the period of printing because of evaporation of water that is the most volatile component of the polymerization solution. This change in the acrylamide concentration may lead to spontaneous polymerization of the solutions in the wells after just a few print runs.

Under ideal conditions, right after dispensing on a substrate, the droplets form an array and are expected to rest in their positions until polymerization. The polymerization results not only in formation of gel, but also in formation of chemical bonds between the gel and the substrate. So, after the polymerization, the droplets lose their mobility. Humidity control systems built into some commercial arrayers destabilize local humidity above the substrates, e.g. above slide holders. Despite the humidity control, oscillating humidity leads to migration of the droplets from their intended positions and non-uniform droplet composition.

In the context of making gel-element microarrays, evaporation is an issue relevant to a contact-printing technology as opposed to the technology of mask-guided photopolymerization in a continuous layer. However, in the context of making biochips, which includes fabrication of gel-element arrays as one of the steps, evaporation is an issue with both above-mentioned technologies because the latter one uses contact printing as a method of dispensing biomolecular probes on pre-fabricated gel-element arrays, and contact printing implies using a source microplate (it contains the solutions to be dispensed) exposed to ambient air.

### SUMMARY

Two issues arise in preparation of microarrays, in particular by use of commercial arrayers-evaporation in the source plate and evaporation from droplets after printing. Small (of the order of 1 nl) droplets containing components for the microarray, are dispensed from a source plate on substrates such as glass slides (“printing”). The plurality of substrates, e.g. glass slides, are then stored until a batch is ready for polymerization, e.g. by ultraviolet light (UV). Lack of humidity control in the source plate as well as during dispensing of the drops (“printing”) and storage of the arrays on substrates is undesirable because oscillation of humidity causes non-uniform, non-reproducible, array (microchip) quality.

Methods and compositions are disclosed herein to generate an environment during production, storing and polymerization of the microarrays that is in equilibrium with regard to vapor pressure, that is, vapor pressure in the gaseous environment of the production system. All environments are controlled because the polymerization mixture of the droplets, minus glycerol, is used to saturate the solution in the source plate and the chamber (cassette) used for storing cassettes after printing prior to and during polymerization. The vapor pressure of water in the gas is maintained the same as in the droplets (Rault's Law). O₂ is also excluded. To produce the vapor, air flows over the solutions from the polymerization mixture.

A method to control evaporation when preparing gel-based microarrays using a polymerization mixture includes the steps of:
(a) including the components in the polymerization mixture whose equilibrium vapor pressure is similar or lower than glycerol, that is excluding components from the mixture that have an equilibrium vapor pressure higher than that of glycerol; and
(b) placing gel droplets in a container in which polymerization by ultraviolet light occurs. The container has a chemically neutral gas replacing oxygen and the neutral gas vapor transports acrylamide from the mixture to the droplets and maintains equilibrium. An incubation step in the microarray fabrication protocol is combined with the polymerization step.

One or more substrates including supported droplets, before and during polymerization, are placed into the sealed container under oxygen free inert atmosphere with controlled humidity. The container is filled with one of the following gases: N₂, Ar, CO₂, and gaseous media are continuously or periodically restored in the container of the substrates.

A method of improving fabrication of a polymer-based microarray includes the steps of:
(a) including in a polymerization mixture (solution) components that have equilibrium vapor pressure similar or lower than glycerol, that is excluding from a polymerization solution all components that have equilibrium and vapor pressure higher than that of glycerol, but keeping water;
(b) following a sample dispensing step onto substrates (“gel drop”), restoring a predetermined composition of the polymerization mixture by exposing the substrates to a predetermined atmosphere in an air-tight cassette, wherein the atmosphere contains acrylamide;
(c) maintaining the atmosphere of (b) throughout polymerization by ultraviolet light; and
(d) rehydrating the source plate by restoring water concentration in the polymerization mixture by exposing the source plate to a predetermined humidity.

Optionally, in a “gel drop” method, there is no acrylamide used. (See Materials and Methods). However, if acrylamide is used in the vapor phase, microarrays of uniform and improved quality result. The atmosphere equilibrates with the gel drops and the method is reproducible. In this optimum environment gel does not swell, shrink or lose its constitution. Acrylamide is forced into the droplets, polymerizes and reaches equilibrium. This method is referred to as “restoration techniques.”

The optimum “right” atmosphere is atmosphere that is in equilibrium (in terms of vapor pressures) with the polymerization mixture in the bath that serves as a source of volatile chemicals such as acrylamide in the apparatus for incubation/polymerization (FIG. 1). The composition of the mixture in the bath determines (according to Raoult's Law) the vapor pressure of acrylamide and other chemicals inside the container; in its turn, the vapor pressure of acrylamide determines the concentration of acrylamide in the droplets. In other words, the flow of carrier gas provides a means for transport of acrylamide from the mixture in the bath to the droplets. The transport stops as soon as equilibrium in the entire system is reached.

Biochips are prepared by the methods disclosed.

The methods, apparatuses, and compositions disclosed herein are useful for improved manufacturing of polymer-element microarrays by co-polymerization or any other suitable technique. In comparison to other processes used for manufacturing of polymer-element (gel) microarrays, the methods, apparatuses, and compositions disclosed herein offer high reproducibility of microarrays; substantially relax restrictions on duration of a printing run, which is essential for scaling up the production capacity and manufacturing of complex microarrays containing hundreds and thousands of biomolecular probes. In addition to microarray fabrication, the methods, apparatuses, and compositions disclosed herein are useful for fabrication of arrays of microlenses for optoelectronic's and adaptive optics, e.g., tunable lenses.

### Definitions as Used in This Disclosure are:

Array, Microarray: refers to molecules generally connected to a matrix or support (substrate) in a specific arrangement relative to each other, also known as DNA microarray, DNA array or peptide array.

Arrayer: is a robotic system used in microarray manufacturing for dispensing solutions of biomolecular probes (oligonucleotides, proteins and the like) onto microarray substrates.

Biochip: is a set of (array of) biological molecules (called probes) attached in an appropriate order to a substrate/support or matrix. Also known as a chip, DNA chip or peptide chip; includes array of biological molecules such as DNA fragments, peptides, proteins, lipids, and tissues connected to a substrate.

Biological microarray, biochip, chip: arrangement typically on a glass, plastic, filter, or silicon wafer, of DNA fragments, peptides, proteins, lipids or other biological compounds deposited or synthesized in a predetermined spatial order.

Cassette: is an airtight enclosure specifically designed for processing multiple microarray substrates at the steps of incubation and polymerization. The enclosure is provided with UV-transparent window(s) for illuminating the substrates and appropriate fittings that allow one to control atmosphere inside the enclosure.

Gel-element: microarray composition for immobilization of molecules in polymer carriers, wherein the polymer layer may be a three dimensional gel.

Gel-element microarrays: may be used as substrates for biochips. A gel-element microarray becomes a biochip when oligonucleotide (or protein) probes are immobilized in the elements of the array. A method of manufacturing gel-element microarrays refers to manufacturing of biochips as an application of the method.

Polymerization mixture: (synonymous with “polymerization composition”) is a liquid mixture (generally a solution) of all the reagents required for formation, under certain conditions, of a cross-linked polymeric matrix (gel). In particular, “certain conditions” may mean irradiation with ultraviolet light. An example of “cross-linked polymeric matrix” is polyacrylamide gel. The polymerization mixture may include additives that modify the properties of gel that can be obtained by polymerizing the mixture. It is generally assumed that such additives neither interfere with polymerization or promote it. With regard to biological microarrays (biochips), examples of such additives are oligonucleotides and proteins.

Sample: refers to a polymerization mixture that includes a specific additive (such as oligonucleotide or protein) which differentiates this particular mixture from other “samples” that are generally assumed to comprise additives different from the one in question. For instance, a microplate prepared for printing microarrays is a “source microplate loaded with the samples”. Solutions in the microplate wells may differ from one another although all of them are capable of polymerizing under certain conditions.

A source microplate: is e.g., a plastic microtiter plate (the types most common in microarray have wells of 50 microliters each) loaded with solutions (samples) to be dispensed on microarray substrates such as glass slides.

### BRIEF DESCRIPTION OF THE DRAWINGS

FIG. 1 is a schematic of preparing a microarray.

FIG. 2 Gel Pads—No Restoration. The upper two rows are printed with a 150-micron pin. The gel element diameter is about 100 microns. Printing was from right to left. The smaller the droplet, the faster it is depleted of acrylamide. For this reason the droplets printed first (at the right) failed to polymerize, and the intensity of fluorescence decreases from left to right. Evaporation of acrylamide results in less dense gel, which means lower probability of oligonucleotide immobilization and lower fluorescence intensity. The lower row of gel pads was printed with a 300-micron pin. The gel element diameter here is about 250 microns. The ratio of surface to volume is inversely proportional to the droplet diameter. Therefore, the larger the droplet the slower the process of acrylamide depletion. It is evident that the 300 microns droplets have higher temporal stability.

FIG. 3 Gel Pad Array After Restoration. This array of gel pads (containing a labeled oligonucleotide) was printed with a 150-micron pin and polymerized after 1-hour restoration. Because the substrate (slide) in this case was more hydrophobic, the droplets diameter was between 80 and 90 microns. Yet, the array is more uniform then in FIG. 1 top 2 rows. The relative standard deviation of the fluorescence intensity is about 6%. Recording was done using a ScanArray 3000 microarray scanner (Packard Bioscience).

### DETAILED DESCRIPTION

A system to produce a microarray includes having a polymerization mixture in a source plate from which samples are dispensed (“printed”) onto substrates, for example by a robotic arrayer. The arrayer is in an enclosure. After printing, the substrates with the “gel drops” are incubated and polymerized in a cassette.

Methods and compositions described herein prevent evaporation throughout the system by controlling the environment thereby producing improved microarrays.

Improved methods, apparatus, and compositions for fabricating polymeric elements at pre-determined locations on a solid substrate used in synthesizing biological microarrays are described. Glass or plastic slides are examples of suitable substrates. The methods and compositions of the present disclosure provide more homogeneous (referring to porosity) gel-elements. Polymer-based microarrays (gels) also involve fabrication of three dimensional (3D) polymeric layers at pre-determined locations on a solid substrate.

Stratification of the gaseous medium in the container used for restoration of chemical composition of the droplets occurs under general operating conditions, because acrylamide vapor is more dense than air, and air is more dense than water vapor. Placing the arrays of droplets in a sealed container, where the only environmental parameters under control are the concentrations of oxygen and water vapor, also substantially depletes the droplets of acrylamide unless certain additional conditions are met.

Methods and compositions disclosed herein provide conditions for reliable and reproducible polymerization of droplets by controlling the concentrations of some or all of the essential chemicals throughout the manufacturing procedure of microarrays. Migration of droplets after dispensing them on microarray substrates is prevented or minimized regardless of the dispensing technique (e.g. contact or non-contact printing) and substrate material or surface coating used in the fabrication process. The lifetime of the source plates is extended considerably by reducing the spontaneous non-uniform polymerization in the wells.

For array printing, components that are considerably (>50%) more volatile than glycerol are removed from printing solutions, except water. Relative humidity inside the arrayer enclosure is maintained at a steady level, about close to, but generally not exceeding the equilibrium pressure of water vapor over the solution in the source plate.

As soon as substrates with droplets from the atmosphere saturated with acrylamide vapor are removed, the process of acrylamide depletion starts all over again. This doesn't necessarily mean that such droplets will inevitably fail to polymerize, but the final properties (such as porosity) of the gel elements may vary from batch to batch depending on how long the substrates were exposed to ambient air before the polymerization, and what was the intensity of inert gas flow during the polymerization. This uncertainty can be eliminated by running polymerization in the same cassette and atmosphere (of saturated acrylamide vapors) as those used at the step of incubation preceding the polymerization.

For array incubation, following the printing step, the required compositions of the polymerization mixture are restored by exposing the arrays to an optimal atmosphere in an air-tight container. The optimal “right” atmosphere is atmosphere that is in equilibrium (in terms of vapor pressures) with the polymerization mixture in the bath that serves as a source of volatile chemicals such as acrylamide in the apparatus for incubation/polymerization.

For array polymerization, the optimal atmosphere throughout the polymerization step is maintained. For plate or microarray re-hydration, water concentration in the samples is restored by exposing the source plate(s) to an optimal humidity, as disclosed herein.

Compositions of mixtures used for printing may not include components that are required for synthesis of a polymer layer (e.g. polyacrylamide gel), but are too volatile under conditions of printing. Chemical composition of droplets as required for polymerization is then reconstituted at an incubation step that follows printing.

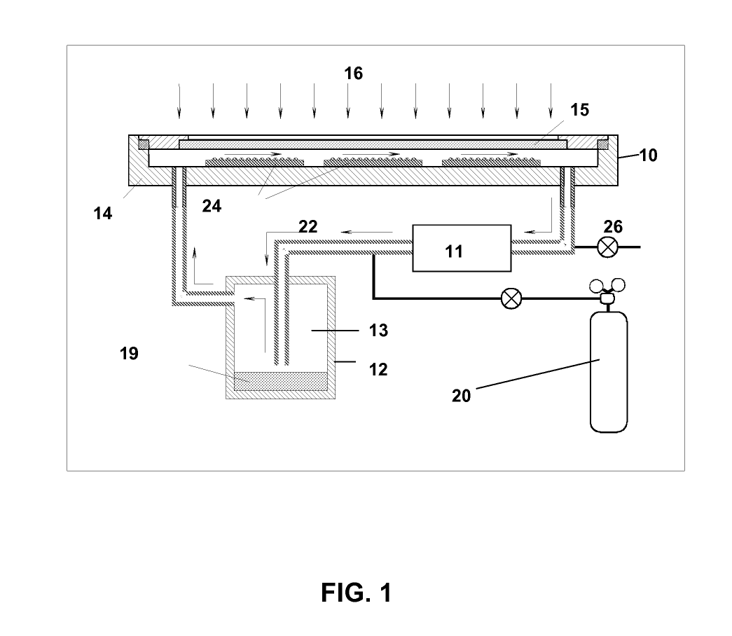

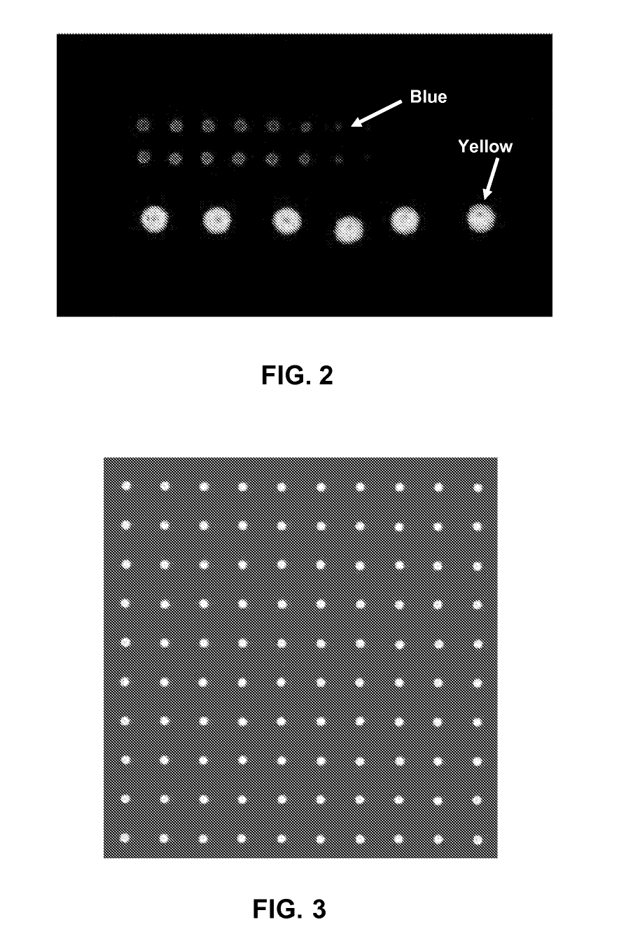

Methods to facilitate vapor-phase transport of chemicals during incubation and to provide stratification-free atmosphere include recirculation of gas in the system using a pump of chemically inert design. (FIG. 1)

Methods to minimize loss of monomers include performing the UV polymerization in the same gaseous environment as the one used at the incubation step. The disclosed methods provide procedures and recommendations that eliminate migration of droplets throughout the fabrication process. Eliminated migration results in improved geometrical stability. Migration-safe humidity levels are those not exceeding the equilibrium humidity for the given concentration of water in the polymerization mixture (or samples). The equilibrium humidity can be roughly estimated using Raoult's Law, or found in the literature (see, for example, Eric et al. 1999), or determined experimentally. For example, for the mixture of 65% w aqueous solution of glycerol the upper limit of the migration-safe humidity range is about 52%. Additional steps of source plate re-hydration are introduced to stabilize rheology of the samples.

Steps described herein to modify microarray manufacturing methods and apparatus to control evaporation include: excluding from the composition of the polymerization mixture some components that have equilibrium vapor pressure considerably (e.g., >50%) exceeding that of glycerol (one of the most abundant components of an embodiment of a gel formulation) i.e., is more volatile. For example, for fabrication of arrays composed of polyacrylamide gel, as in U.S. Patent No. 2004/0053298, the polymerization mixture includes glycerol, bis-acrylamide, and, optionally, a biomolecular probe such as oligonucleotide or protein, but not acrylamide.

Another step to modify microarray manufacturing methods and apparatus includes, for example following the sample-dispensing step, the arrays of droplets are placed in an airtight cassette of a design that allows ultraviolet polymerization of the gel. The cassette is purged of oxygen with a flow of a chemically neutral gas (such as nitrogen), which also serves as a carrier gas for supplying the cassette with the vaporized components of the polymerization mixture (in the case of polyacrylamide gel, these are glycerol, water, bis-acrylamide, and acrylamide.)

The vapors are generated by blowing the carrier gas over a bath filled with the polymerization mixture formulated for making a gel. This incubation is maintained for a period of time sufficient to reach equilibrium (in terms of chemical composition) between the solution in the bath and the droplets on the substrates in the cassette.

Actively re-circulated carrier gas promotes equilibrium conditions inside the container which is essential for uniform processing of the substrates and substantially reduces the time required for incubation. The volume of the container determines the amount of chemicals present inside the container in the vapor phase. Substrates are kept in an oxygen-free equilibrium atmosphere both before and during polymerization.

The composition of droplets of polymerization mixture printed on microarray substrates are restored by exposing the substrates to an atmosphere of inert gas that is in thermodynamic equilibrium with the polymerization mixture. Substrates with droplets are preferably kept in an atmosphere controlled in terms of both oxygen and the volatile chemicals not just prior to polymerization but also during polymerization. Transport of the volatile chemicals (mainly the monomer, acrylamide) from the bath with polymerization mixture to the droplets are achieved by re-circulating chemically neutral carrier gas. A well-substantiated criterion is provided herein for calculating the amount of polymerization mixture required for ensuring equilibrium atmosphere inside the cassette.

When the incubation step is completed, the droplets are polymerized by exposing them to ultraviolet light with a center emission wavelength of about 312 or 365 nm.

Another step to modify the microarray manufacturing methods and apparatus includes, for example, that during the microarray fabrication, the source plates containing the polymerization solutions lose water due to evaporation, so that upon completion of the fabrication, the source plates are put into an environment with controlled humidity for re-hydration. The relative humidity is chosen to be approximately equal to the molar fraction of water in the samples.

A non-volatile solute (or dissolved substance) is glycerol, and the volatile solvent is water. “Mole fraction” (or molar fraction) is in essence the relative abundance of water molecules in comparison to the total number of molecules available in the solution. In this disclosure, one may consider mostly water and glycerol because acrylamide and other chemicals present in the polymerization mixture constitute only a small fraction of the total number of molecules available. According to Raoult's Law, the vapor pressure of a solution of a non-volatile solute is equal to the vapor pressure of the pure solvent at that temperature multiplied by its mole fraction.

Another step to modify the microarray manufacturing process and apparatus includes extending the incubation step in the microarray fabrication protocol so it is combined with the polymerization step to preserve the chemical composition of array elements throughout the polymerization process.

### EXAMPLES

### Example 1: Improving Sample Dispensing Step

During sample dispensing, components that have equilibrium vapor pressure considerably (more than 50%) exceeding that of glycerol (are more volatile) are excluded from the composition of samples. Similarly, components whose equilibrium vapor pressures are equal or lesser than that of glycerol are included. For example, for fabrication of arrays composed of polyacrylamide gel, the samples generally include glycerol, bis-acrylamide, and, if needed, a biomolecular probe such as an oligonucleotide or a protein or a peptide, but not acrylamide. Water, however, is included because otherwise uncontrolled growth of droplets volume due to moisture in the ambient air would cause droplet migration from their intended locations on the substrate. Relative humidity inside the arrayer enclosure during a printing run is maintained at a substantially steady level that generally does not exceed the equilibrium pressure of water vapor over the solution in the plate (as determined by the molar fraction of water in the polymerization composition).

### Example 2: Methods to Improve Incubation During Microarray Fabrication

Following the sample-dispensing step, the arrays of droplets are placed in an airtight enclosure (“cassette”) of the array design module that allows UV polymerization of gel (FIG. 1). The cassette 10 includes an airtight enclosure 14 and is connected to a pump 11 and a chamber 12 that contains a bath 13 filled with a mixture of glycerol, water, bis-acrylamide, N,N-Methylene-bis-acrylamide and acrylamide taken in proportion equal to that of the polymerization composition 19 required for preparation of gel with desired properties. After loading the cassette 10 with microarrays (substrates 24), the cassette is flushed of air using a chemically neutral gas 20 such as nitrogen. The purpose of flushing is to remove oxygen that affects polymerization of acrylamide. Then the pump is activated to provide a constant gas flow 22 in the closed contour formed by the components of the system. The pump capacity and piping are chosen to provide conditions for a weakly turbulent flow in both the chamber and the cassette. The methods and steps described herein are intended to create in the cassette, a stratification-free gas environment, in which the chemicals are generally present in equilibrium concentrations determined by the composition of the solution in the bath. Accordingly, the volume of the solution should be large enough to ensure that the mass of each of the components in the liquid phase exceeds at least ten times the mass of that component in the vapor phase. The time of incubation required for reaching equilibrium (in terms of chemical composition) between the solution in the bath and the droplets on the substrates in the cassette is determined experimentally. For example, for a composition needed for synthesis of polyacrylamide gel and the total volume of the system is about 1 liter, the time of incubation is close to about one hour.

As shown in FIG. 1, in an embodiment, the substrates are enclosed within an air-tight enclosure 14. The enclosure has a quartz window 15 to permit UV light 16 for polymerization initiation. A pump 11 re-circulates the air from the enclosure along with an inert gas 20 from a cylinder. The inert gas 20 can be nitrogen. The inert gas 20 is passed through a container 12 that has the polymerization composition 19. The polymerization container is connected to the enclosure 14 where the substrates 24 are positioned.

### Example 3: Polymerization

The polymerization of the droplets on the substrates is carried out by exposing them to UV light, e.g. with a center emission wavelength of about 312 nm or 365 nm. The light is switched on when the incubation step is completed. The polymerization composition used for printing gel-element microarrays includes relatively volatile components that can get depleted due to evaporation during the process of printing. This may lead to irreproducibility of gel-elements properties and spontaneous polymerization of the mixture in the source plate. The materials and methods described herein present a technique for restoring the original composition of the droplets dispensed on the microarray substrates prior to the step of UV polymerization. An apparatus for implementing the technique is also disclosed.

### Example 4: Re-hydration of the Source Plate(s)

During the print run, the source plate containing the polymerization solutions generally loses some water via evaporation. Upon completion of printing, the source plates are put in environment with controlled humidity for re-hydration. The relative humidity level (expressed as a ratio of the actual pressure of water vapor to its pressure under saturation conditions) is chosen to be equal to the molar fraction of water in the samples.

### Example 5: Raoult's Law, Rehydration

If a plate of water is placed in an airtight container, after a while, part of water evaporates, but then evaporation stops because the system (water+gaseous medium above it) reaches equilibrium. Under equilibrium, losses of water due to evaporation are compensated by condensation of water vapor back in the plate, and water vapor inside the container is said to be saturated. Since relative humidity is defined as a ratio of water vapor pressure under current conditions to that under saturation, at saturation there is 100% Relative Humidity.

If pure water in the plate is replaced with a solution of glycerol in water, the conditions of equilibrium will change to reflect lower abundance of water in the solution. According to Raoult's Law, if water molecules constitute only 80% of the total quantity of molecules in the solution (which means that molar fraction of water is 0.8), then the pressure of water vapor in the atmosphere inside the container will be equal to only 80% of the saturated vapor pressure over pure water. In other words, this means that for aqueous glycerol humidity inside the container will drop from 100% to 80%. The above considerations form a rationale of the method used for restoring water concentration in the source microplate(s).

Plates can be re-hydrated by placing them into an environment where relative humidity is maintained at the level numerically equal to the molar fraction of water in the initial composition of the samples. Since the samples have been depleted of water during the preceding print run, the balance between evaporation and condensation will be shifted toward condensation until the solutions in the wells of the plate reach equilibrium with the atmosphere. At that point, the concentration of water in the samples should return back to its initial value.

### Example 6: Microlens Fabrication

Fabrication of microlens arrays is a well-known area of research and development in optics and optoelectronics. In particular, microlenses are used to improve light collection efficiency of photosensors, and, accordingly, arrays of microlenses are used for the same purpose in the case of multielement photosensors. An example of multi-element photosensors is CCD sensors widely used in digital cameras as well as in numerous other applications in science and technology. In some applications, there is a need for tunable microlenses (or tunable microlens arrays). In most cases tunability implies that there is a mechanism that allows changing focusing properties of the lens (characterized by focal distance parameter). In particular such tunability may be provided by a certain mechanism that allows changing the curvature of lens surface. There are a number of approaches to synthesis of microlens arrays. In many cases such lenses are made of transparent polymers. Polyacrylamide is a well-known material for fabrication of lenses, in particular, contact lenses. Gel elements fabricated by the method disclosed in this invention are lens-like in shape. Moreover, they can actually work as lenses. The gel elements are inherently hygroscopic. The curvature of the gel elements varies depending on the amount of water absorbed by the gel element. In fact, this feature provides opportunity to control focusing properties of the gel elements, which, therefore, can be considered as tunable microlenses.

Three dimensional polymeric layers can also be employed in optoelectronics and optics, for example, for microlens array synthesis. Gel is a preferred polymer. An array of gel elements fabricated by the methods describe can be readily considered an array of microlenses due to the shape of gel elements and their optical properties (such as transparency and refractive index). Choice of polymer composition is not limited to the use of polyacrylamide.

### MATERIALS AND METHODS

### Digital Imaging in Optical Microscopy

Microlens Arrays: Microlens arrays (also referred to as microlenticular arrays or lenslet arrays) are used to increase the optical fill factor in CCDs, such as interline-transfer devices, that suffer from reduced aperture due to metal shielding. These tiny lens systems serve to focus and concentrate light onto the photodiode surface instead of allowing it to fall on non-photosensitive areas of the device, where it is lost from the imaging information collected by the CCD.

A typical lenslet placement scheme has tiny optical lens in strategically placed over the dye layer and metal light shield of a photodiode. The lenslets are either grown in parallel arrays during the CCD fabrication process or manufactured out of a material such as quartz and placed on the array surface during packaging. Each lenslet is a high quality optical surface containing refractive elements ranging in size from several hundred to around 10 microns in diameter, depending upon the application. Lens quality is so good that microlenses are physically equivalent to an ordinary single-element lens.

Addition of microlens arrays to CCD photodiodes can increase the optical fill factor by up to three times that realized without the tiny optical components. Increasing the fill factor yields a corresponding increase in the sensitivity of the photosite. Microlens arrays provide a substantial increase in performance of interline-transfer CCD imaging arrays that have lateral overflow drains and a sizeable amount of shielded pixel space. These devices typically suffer from reduced optical fill factors because of reduced active pixel area compared to total pixel size.

An interline-transfer CCD pixel pair is, one equipped with a microlens to concentrate light into the photodiode, while the other must absorb incident light rays without the benefit of optical assistance from a microlens. Incident photons that strike the microlens are directed into the photodiode by refraction through the glass or polymer comprising the microlens. The photodiode without a microlens collects a significantly lower portion of incoming photons, because those that impact on shielded areas (the exposure gate and neighboring structures) are not useful in charge integration. The optical fill factor of interline CCDs can be reduced to less than 20 percent by fielded vertical transfer shift registers. With the microlens array, the fill factor can approach 100 percent, depending upon manufacturing parameters.

Organization of the cone of light reaching the microlens surface depends upon the optical characteristics of the microscope or camera lens used to direct light to the CCD. Also, polysilicon gate thickness heavily influences the ability to collect light by the photodiode positioned beneath the gate structure. Microlens arrays are fabricated using reflow techniques on resist layers to achieve numerical apertures ranging from 0.15 to 0.4 with short focal lengths and corresponding lens diameters of 20 to 800 microns. The fill factor of a microlens array is strongly dependent upon the manufacturing process used to create the array. Glass lenses of somewhat lower (0.05 to 0.2) numerical aperture are also utilized. Lower numerical aperture microlenses have fewer optical aberrations with significantly longer focal lengths.

Disadvantages encountered with microlens are far outweighed by sensitivity of devices having these optical components in place. One of the primary difficulties occurs when light rays from the outer portions of a pixel are focused onto an adjacent lens (and subsequently onto the detector photodiode) resulting in mis-registration. In addition, when detector pixel size reaches the diffraction limit of the microlenses, the pixels become overfilled leading to inaccurate measurements. As photodiodes become smaller, the problems associated with producing quality microlenses increase. Higher quality microlenses are needed to produce images on these arrays, but spherical aberration then becomes a problem. Adding microlenses to CCDs increases the number of processing steps, and the uniformity of the lens array is a variable that can often cause problems during fabrication.

Saturated vapor pressure at 25 C: water-23.76 mm Hg, acrylamide-0.007 mm Hg, glycerol-0.000268 mm Hg

Formula for the droplets:
65% (w/w) glycerol (Sigma-Aldrich Inc., St. Louis, Mo., US)
5% (w/w) acrylamide /N,N′-methylenebisacrylamide in a 19:1 proportion (prepared using a 40% Acrylamide/Bis Solution, 19:1, Bio-Rad, Hercules, Calif., US)
0.25 mM oligonucleotide probe (which is, generally, different for each well of the source microplate)
0.035 M sodium-phosphate buffer (pH 7.25)

Acrylamide may be left out of this solution totally (but bis is still required because it is much less volatile than acrylamide). One may add acrylamide to the droplets via vapor phase at the restoration step. A standard commercial solution contains both acrylamide and bis.

Restoration Solution using the following recipe:
65% (w/w) glycerol (Sigma-Aldrich Inc., St. Louis, Mo., US)
5% (w/w) acrylamide /N,N′-methylenebisacrylamide in a 19:1 proportion (prepared using a 40% Acrylamide/Bis Solution, 19:1, Bio-Rad, Hercules, Calif., US)
0.035 M sodium-phosphate buffer (pH 7.25)

As indicated, this mixture is prepared using a standard 40% aqueous solution of acrylamide and N,N′-methylenebisacrylamide (“Bis”) which are present in the solution in proportion of 19:1. For example, 100 g of the restoration solution should contain 5 g of acrylamide and bis by dry weight. Accordingly, to prepare this solution one should take 5 g/0.4=12.5 g of the 40% acrylamide/Bis solution.

The latter is a standard reagent for preparing electrophoretic gels and is readily available from a number of vendors and is convenient to use (e.g. one doesn't have to deal with dry acrylamide that is a hazardous substance). However, this doesn't mean that the restoration solution cannot be prepared using some different approach. Also, the final concentrations of acrylamide and bis may differ from 4.75 and 0.25%, respectively. Variations of these concentrations will result in formation of gel of somewhat different properties which doesn't necessarily imply dramatic degradation of microarray performance. For instance, in our practice we varied the concentration of acrylamide+bis in the range of 4 to 5%.

Finally, the restoration solution may be prepared without bis, because volatility of bis is much lower than that of acrylamide. For this reason, the content of bis in the drops of the polymerization solution printed on the microarray substrates doesn't change much in the process of array fabrication. The feasibility of fabricating chips using a bis-free restoration solution has been proved experimentally.

### DOCUMENTS CITED

These documents are incorporated by reference to the extent they relate or explain materials or methods disclosed herein.

Eric et al., Journal of Solution Chemistry, (1999) (10): 1137-1157.

U.S. Pat. No. 6,465,174 entitled: “An Improved Method for Photopolymerization of Microchip Gels”.

U.S. Pat. Pub. No. 2004/0053298 Al entitled: “Composition of Polymerising Immobilisation of Biological Molecules and Method for Producing Said Composition”.

U.S. Pat. No. 6,171,883

U.S. Pat. No. 6,625,351

We claim:

1. A method to control evaporation during preparation of gel-based microarrays using a polymerization mixture, the method comprising:
(a) including components in the polymerization mixture whose equilibrium vapor pressure is similar or lower than glycerol, that is, excluding components from the polymerization mixture that have an equilibrium vapor pressure considerably higher than that of glycerol;
(b) dispensing gel drops from the polymerization mixture; and
(c) incubating and polymerizing gel drops in a cassette in a vaporous environment consisting of the polymerization mixture excluding glycerol.

2. The method of claim 1 further defined as wherein the vapor of a chemically neutral gas replaces oxygen and transports acrylamide from the mixture to the gel drops.

3. The method of claim 1 wherein an incubation step is combined with the polymerization step.

4. The method of claim 1 wherein the gel-based microarrays are biochips.

5. A method of improving fabrication of a polymer-based microarray, the method comprising:
(a) excluding from a polymerization solution substantially all components that have equilibrium vapor pressure considerably higher than glycerol;
(b) restoring a predetermined composition of the polymerization solution by exposing the arrays to a predetermined atmosphere in a chamber, wherein the atmosphere contains acrylamide;
(c) maintaining the predetermined atmosphere throughout polymerization by ultraviolet light; and
(d) rehydrating a source plate by restoring water concentration in the polymerization solution by exposing the source plate to a predetermined atmosphere

6. An apparatus for improving fabrication of a polymer-based microarray, as in FIG. 1.

7. A microarray module comprising:
(a) an ultraviolet light permissive enclosure for receiving the microarray, the enclosure connected to a pump;
(b) a chamber associated with the enclosure, the chamber adapted to provide an optimal vapor pressure, wherein the chamber comprises one or more constituents present in a polymerization mixture; and
(c) a source of inert gas functionally associated with the chamber.

8. The microarray module of claim 7, wherein the enclosure is a cassette.

9. The microarray module of claim 8, wherein the cassette is air-tight.

10. The microarray module of claim 6, wherein the pump introduces the inert gas.

11. A biochip prepared by the method of claim 1.

12. A biochip prepared by the method of claim 5.
